(19) United States
(12) Patent Application Publication
(10) Pub. No.: US 2022/0399115 A1
(43) Pub. Date: Dec. 15, 2022

(54) SYSTEM AND METHOD FOR PREDICTION OF DISEASES FROM SIGNS AND SYMPTOMS EXTRACTED FROM ELECTRONIC HEALTH RECORDS

(71) Applicant: Milagro AI Care Ltd., Hod Hasharon (IL)

(72) Inventor: Alon ALTER, Raanana (IL)

(21) Appl. No.: 17/346,510

(22) Filed: Jun. 14, 2021

(51) Int. Cl. G16H 50/20; G16H 10/60; G16H 70/60; G16H 50/70; G06N 20/00; G06F 40/205

(52) U.S. Cl. CPC G16H 50/20; G16H 10/60; G16H 70/60; G16H 50/70; G06N 20/00; G06F 40/205; G06Q 50/26

(57) ABSTRACT

A method and a system for efficient prediction of diseases is presented. Medical prediction systems usually learn and make predictions based on huge amount of raw clinical data, in the order of tens of thousands of data points which are sparse, episodic, and noisy. This approach requires big computing resources, which are unavailable to most health centres. In the disclosed approach, the dimensionality of the multitude raw data is automatically reduced by applying published disease protocol algorithm and guidelines to the huge number of patient features of raw data stored in EHR (Electronic Health Records), clinical text documents and clinical devices, to obtain few hundred data points which are fed to predicting machine both for the training phase and for the real time disease prediction. Thus, many health centres can benefit from the use of advanced prediction system to improve their performance, using their existing computer resources of.

### TECHNICAL FIELD

The present invention generally relates to medical computer method and system. More particularly it relates to computer aided method for generation medical prediction.

### BACKGROUND ART

Artificial Intelligence in general and deep learning model in particular play increasing role in healthcare data processing. It is being used for diagnostics as well as for prediction of diseases. Predictive analysis tools have become increasingly important both for wellbeing of the patients as well as for the profitability of healthcare providers and for reducing the cost for the healthcare payers.

The input to medical prediction system is comprised of multiple resources and data types such as lab results, medications, coded diagnosis and procedures, and non-structured texts (such as Surgery reports, Radiology reports, patient reported symptoms).

The amount of information for each patient is noticeably big, comprising of tens of thousands of variables, and the information over time is sparse and episodic. For implementing deep learning model, huge number of patients' data must be analysed, so that the computing resources needed are very expensive, and thus most of the healthcare organizations cannot afford such a system.

US Publication number 2014/0278490 A1 by E. M. Holtham “System and Method nor Grouping Medical Codes for Clinical Predictive Analytics” uses an algorithm that groups medical codes to reduce the number of variables for the predictive machine. It does not use all the relevant input data contained in EHR and textual documents, only the medical codes. therefore, the quality of the prediction is limited.

US Publication Number 2018/0247193 A1 “Neural Network Training Using Compressed Inputs” refers to a neural network used for the analysis of images obtained from imaging instruments. It reduces the input data by compressing the images using Lossy compression algorithms with various levels of data loss.

US publication Number 2019/0034591 “System and Method for Predicting and Summarizing Medical Events from Electronic Health Records” discloses a prediction method that uses all the data found in the EHR for each patient both for the training phase and to the prediction phase. Therefore, there are millions of input points to the prediction processor, so that huge computer resources are required, resources available in big medical centers.

Hence, there is a need for an affordable, cost effective predictive system that can be used by all healthcare organizations, including medium and small clinical institutes.

### SUMMARY OF INVENTION

The prediction method described in this invention is comprised of two phases. The first phase is the learning phase, where a Deep Learning Predictor model is trained for predicting of a specified disease. In the second phase, the Operation Phase, the Deep Learning Predictor is executed whenever new data is received about a patient. Note that there is a dedicated Deep Learning Predictor for each disease. So, when new data is obtained, all Deep Learning Predictors are executed.

Nowadays big computers resources are required to execute deep learning algorithms when the input includes tens of thousands of variables. The method disclosed reduces the number of variables entering the deep learning algorithm to few hundreds by applying reduction rules based on known published clinical guidelines and practices.

The method described extracts relevant information from Electronic Health Records (EHR) which includes coded diagnoses, laboratory measures, ICU monitors parameters, as well as text documents. The extracted information is being translated into standard medical codes used by the system. These medical codes are going through a reduction process, a process that uses known clinical practices, that results in smaller number of meaningful medical variables which are then mapped to time stamped Model Vector which is processed by the Deep Learning Predictor. The number of variables enters said predictor is in the order of few hundreds.

### BRIEF DESCRIPTION OF DRAWINGS

FIG. 1 shows general flowchart of the system processing for prediction.

FIG. 2 presents detailed flowchart of the system processing.

FIG. 3 contains examples of code reductions.

FIG. 4 contains an example of Model Vector.

### DETAILED DESCRIPTION

The invention will be described more fully hereinafter, with reference to the accompanying drawings, in which a preferred embodiment of the invention is shown. The invention may, however, be embodied in many different forms and should not be construed as limited to the embodiment set forth herein; rather this embodiment is provided so that the disclosure will be thorough and complete, and will fully convey the scope of the invention to those skilled in the art.

Input variables are also referred to as features. The set of all input variables is referred to as the input space, and the number of the input variables is referred to as the dimension of the input space. The variables are represented by codes so that code reduction is equivalent to variable reduction and to dimension reduction. Note that the input variables can be episodic, noisy, sparse, and irregular.

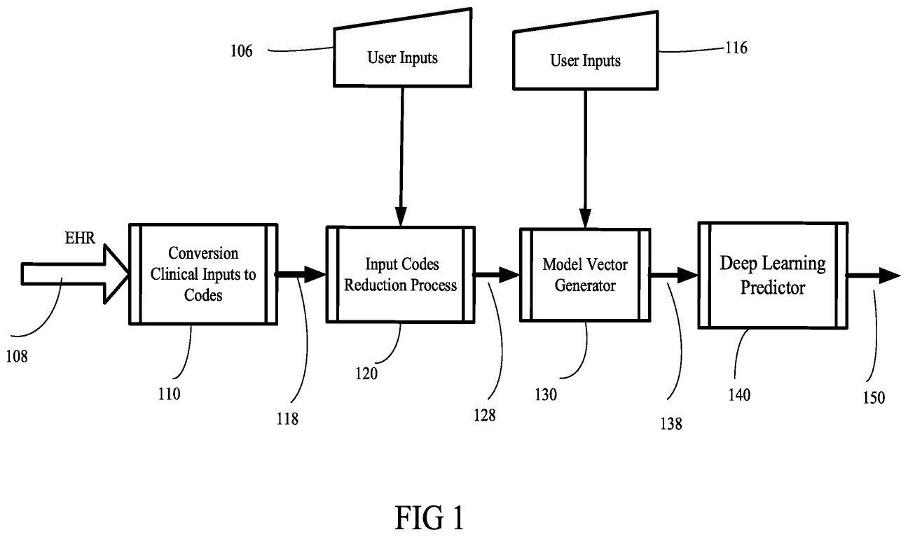

FIG. 1 describes the general data flow and the processes of the disclosed system. The system uses as inputs EHR (Electronic Health Records)—108 which is comprised of structured and non-structured data. The input data 108 is fed to Conversion Clinical Input to Codes process—110 which generates standard codes for input variable—118. The standard codes—118 enter Input Codes Reduction Process (ICRP)—120, that reduces the number of input variables, by applying clinical protocols and guidelines that are published by respected medical publications. This process generates Filtered Medical Information Codes—FMIC 128. Whereas the EHR contains tens of thousands of input variables, the FMIC contains few hundred variables which are meaningful for the predictor, according to the said publications. The FMIC is processed by Model Vector Generator (MVG) 130 that prepares a model vector 138 with reduced dimensionality to be used by the Deep Learning Predictor 140 for training and to produce the required prediction and generates warning for patients on expected diseases 150. The ICRP 120 is controlled by inputs from the user 106, who uses published protocols (e.g., Center of Disease Control guidelines) flow diagrams or other set of rules. The MVG (Model Vector Generator) 130 is controlled by user inputs 116 so the Deep Learning Predictor 140 receives meaningful data for the prediction model. Note, that there is a model vector for each deep learning disease predictor model.

The system is going through a training phase for each disease the user wants to build the prediction for. Thus, the predictor is comprised of multiple models, each targeted to a specific disease. During the training phase, for each disease, the user prepares two groups of EHR record sets, one that contains people with the disease and the other EHR group that is free of the specific disease. At the end of the training phase, the predictor for the disease P_{d} is ready. During operation, whenever new information is obtained from a user, all the predictors in the system operate on the new data and informs about the results of the new prediction data.

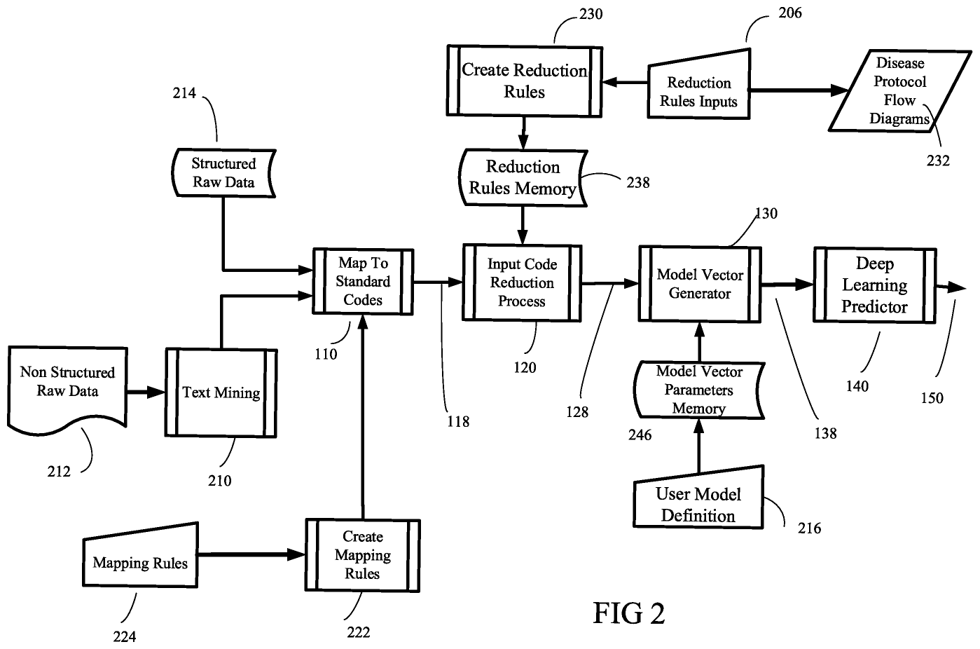

FIG. 2 presents detailed flow diagram of the system processing. The EHR is comprised of Structured Raw Data (SRD) 214 and from Non-Structured Raw Data (NSRD) 212. The SRD—214 includes lab results, coded diagnostics, coded medications, clinical devices signals, and coded procedures. The NSRD—212 includes Radiology reports, surgery reports, progress notes, admission/discharge/case documents. The SRD 214 is stored in coded format. However, these codes can vary from one health care organization to the other. These codes are mapped in process Map to Standard Codes 110 to standard codes used by the system, such as SNOMED. The NSRD—212 is stored in memory as text. This text is processed by Text Mining process 210 that extracts meaningful clinical insights such as diagnoses, surgery procedures, medications taken by patients, patients complaints, etc., which are mapped to the codes used by the system by process 110—Mapping to Standard Codes. This procedure, 110, uses mapping rules which are created by the user—224 via a process 222 which helps the user to create the mapping rules. The mapped codes from all sources—118, enters the Input Code Reduction process 120. A detailed description of the Input Code Reduction Process will be given in a following section.

The output from the Input Code Reduction Process 120, i.e., the Filtered Medical Information Codes 128, enters Model Vector Generator process 130, which generates Model Vector 138 used by the Deep Learning Predictor 140. Detailed description of the Model Vector Generator 130 will follow.

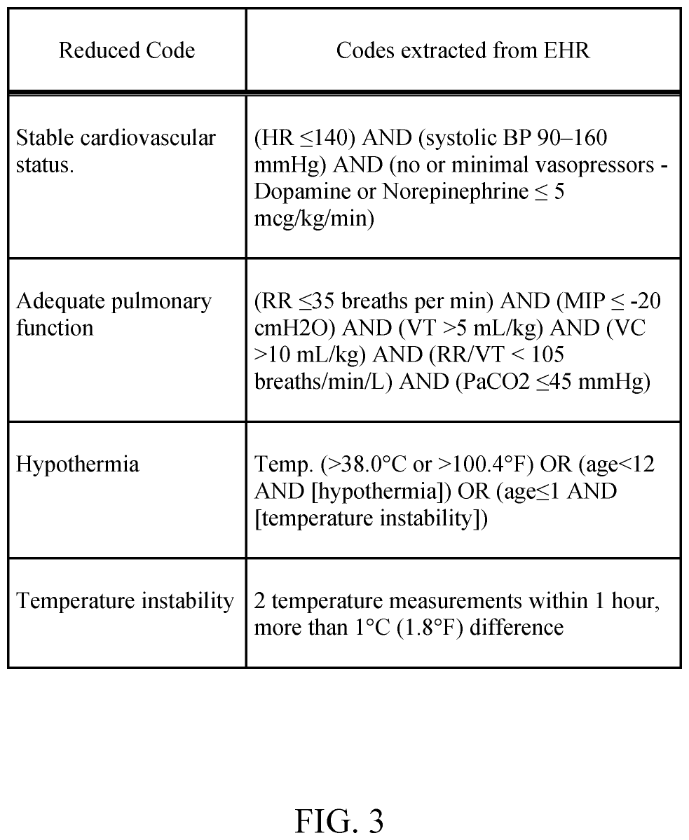

The Input Code Reduction Process 120 generates Filtered Medical Information Codes—FMIC 128, that represent combinations of symptoms/signs which are based on published clinical protocols algorithms. Example of code reduction is shown in the table in FIG. 3. Each raw in the table represents one code reduction transformation. The table has two columns, one describes the reduced code and the other shows the transformation logic executed on the input codes according to the definitions in the disease protocol flow diagrams to derive the reduced code. The transformation is comprised of a set of conditions. For example, Stable cardiovascular status (raw 1) is marked whenever the conditions on the right column are fulfilled, i.e., HR (Heart Rate) is less or equal to 140, and Systolic Blood Pressure is within the range of 90 to 160 mmHg and (Dopamine or Norepinephrine less or equal to 5 mcg/Kg/min). Temperature instability is marked when there are at least two temperature measurements within one hour which differ more than one degree centigrade. Note that the user can generate transformation logic based on his/her experience.

The Input Code Reduction Process 120 uses Reduction Rules which are stored in Reduction Rules Memory 238. These rules are generated by Create Reduction Rules process 230 which is controlled by the user 206 who uses clinical protocols Flow Diagrams 232 as guidelines. Every time a predictor for a new disease is trained, the user adds the reduction rules which are applicable to the new disease. The Reduction Rules for all the Deep Learning Predictors currently in the system are saved in Reduction Rules Memory 238.

A reduction rule is comprised of time dependent logical operations, mathematical operations, and filtering operations. These rules are applied to the incoming patient's raw data. The user, usually a domain expert, such as radiologist, ICU clinician or infection specialist read the protocol guidelines and create rules in non-technical language. As an example, the CDC Site Algorithm for Clinically Defined Pneumonia is defined as follows:

at least one of the following:

### Fever

### Leukopenia

For adults ≥70 years old, altered mental status with no other recognized cause.
AND at least two of the following:
New Onset of purulent sputum or change of character of sputum or increased.
New Onset of worsening cough or dyspnea or tachypnea
Rales or bronchial breath sounds
Worsening gas exchange
Temperature instability (for infants less or equal 1 year old)

### AND

Imaging Test Evidence defined as following: Two or more serial chest imaging test results with at least one of the following New and persistent or Progressive and persistent.
Infiltrate
Consolidation
Cavitation
Pneumatoceles
   Note that each line in the above example is actually a complex rule, as shown in FIG. 3

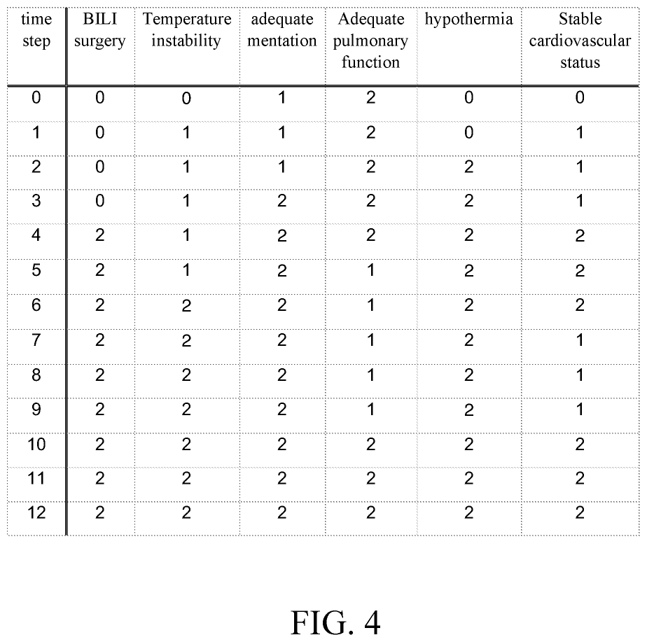

An example of a Model Vector is shown in the table of FIG. 4. Each raw in the table represents time step. The user determines the length of the time step according to the relevant prediction model. Each column represents clinical sign/symptoms obtained after input variables reduction. A value of 0 (zero) denotes that the sign/symptom is not present (e.g., the patient does not have a state of temperature instability), a value of 1 (one) denotes that the sign/symptom is present (e.g., the patient has a state of temperature instability), and a value of 2 (two) indicates that the sign/symptom is relevant, but no data is available (e.g., the data relevant to the patient “Stable cardiovascular status” was not received by the system, thus the patient state of Stable cardiovascular status is not known at this time step). During prediction phase, every time a new data is received, the model vector is updated, so that the last raw is duplicated until the time step of the new information must be updated.

The Model Vector Generator 130 is defined by the user 216 who prepares a set of parameters which are stored in the Model Vector Parameter Memory 246. For each disease to be predicted there is a model.

What has been described above is just one embodiment of the disclosed innovation. It is of course, not possible to describe every conceivable combination of components and/or methodologies, but one of ordinary skill in the art may recognize that many further combinations and permutations are possible. Accordingly, the innovation is intended to embrace all such alterations, modifications, and variations that fall within the spirit and scope of the appended claims.

Furthermore, to the extent that the term “includes” is used in either the detailed description or the claims, such term is intended to be inclusive in a manner like the term “comprising” as “comprising” is interpreted when employed as a transitional word in a claim.

What is claimed is:

1. A method for performing disease prediction from signs and symptoms extracted from Electronic Health Records, the method is comprised of the following steps:
a. reading Electronic Health Records comprised of structured and non-structured data, extracting meaningful clinical data from text documents, converting the clinical data into standard input codes used by the system, and adding time tag;
b. reducing the number of standard input codes to signs and symptoms codes by applying published clinical algorithm;
c. generating disease temporal model vector from the signs and symptoms codes; and
d. applying deep learning disease predictor algorithm to the disease temporal model vector to generate warning on the expected disease.

2. The method according to claim 1, wherein the non-structured data is comprised of text documents from which clinical data is extracted by text mining algorithm.

3. The method according to claim 1, wherein the standard input codes are according to know standards such as SNOMED, Rx, ICD.

4. The method according to claim 1, wherein the published clinical algorithm is the Center of Disease Control (CDC) algorithm, and/protocol or guidelines requested by the client.

5. A method for the reduction of the number of input variables for medical prediction (Dimension Reduction), the method is comprised of the following steps:
a. preparation of sets of logical and mathematical operations that transform plurality of input variables into one variable;
b. applying the sets of logical and mathematical operations to the input variables.

6. The method according to claim 5, wherein the logical and mathematical operations are derived from the Center of Disease Control guidelines or other published and accepted clinical publications.

7. The method according to claim 5, wherein the logical and mathematical operations are derived from experience of the user.
